(19) United States
(12) Patent Application Publication
(10) Pub. No.: US 2023/0157837 A1
(43) Pub. Date: May 25, 2023

(54) SPINAL CERVICAL FUSION CAGE WITH BUILT-IN ANCHORAGES

(71) Applicant: Sylvain Lefebvre, VUILLAFANS (FR)

(72) Inventor: Sylvain Lefebvre, VUILLAFANS (FR)

(21) Appl. No.: 17/534,983

(22) Filed: Nov. 24, 2021

(51) Int. Cl. A61F 2/44; A61F 2/30; B33Y 10/00; B33Y 80/00

(52) U.S. Cl. CPC A61F 2/4455; A61F 2/30771; A61F 2/3094; B33Y 10/00; B33Y 80/00; A61F 2002/3092; A61F 2002/30841; A61F 2310/00023; A61F 2310/00293; A61F 2310/00329; A61F 2310/00005; A61F 2310/00161; A61F 2002/30784; A61F 2002/30985

(57) ABSTRACT

A self-seating spinal cervical cage, including a pair of spaced support members, each respective support member having a first end and a second end, a plurality of teeth extending from each respective first end and each respective second end, a plate member bisecting each respective spaced support member, an array of apertures formed through the plate member, and a porous scaffolding operationally connected to the plate member and defining an open cell pore network. The plate member bisects the porous scaffolding. The plate member and the spaced support members define a unitary titanium body.

### TECHNICAL FIELD

The present disclosure relates generally to a spinal cage implant, and, more particularly, to a spinal fusion cage having built-in anchorages.

### BACKGROUND

The spinal column includes a plurality of vertebrae linked to one another by facet joints and having an intervertebral discs located between each pair of adjacent vertebrae. The facet joints and intervertebral discs allow one vertebra to move relative to an adjacent vertebra, providing the spinal column a range of motion. When the facet joints and/or intervertebral discs become diseased, degenerated, damaged, or otherwise impaired the patient may experience pain or discomfort and/or loss of motion, thus prompting surgery to alleviate the pain and/or restore motion of the spinal column.

One possible method of treating these conditions is to immobilize a portion of the spine to allow treatment. For example, in a conventional spinal fusion procedure, a surgeon restores the alignment of the spine or the disc space between vertebrae by installing a rigid fixation rod and/or a plate between pedicle screws secured to adjacent vertebrae. Bone graft may be placed between the vertebrae, and the fixation rod cooperates with the screws to immobilize the two vertebrae relative to one another so that the bone graft may fuse with the vertebrae. In some procedures, a degree of mobility of the spine is permitted while also providing sufficient support and stabilization to effect treatment.

However, the use of bone screws, rods, and plates, necessitates a substantial amount of hardware left in the body for an extended period of time. In addition to the unavoidable certainty of pain associated with drilling through bone, the hardware involved also incurs a risk of soft tissue injury, infection, dislodgement, fracture, and the like. Thus, there is a need for a spinal cage implant that does not require external bone screws, rods, and/or plates. The present novel technology addresses this need.

### BRIEF DESCRIPTION OF THE DRAWINGS

FIG. 1 is a perspective view of a spinal fusion cage for spinal implant surgery, according to the present disclosure.

FIG. 2 is perspective view of the spinal fusion cage of FIG. 1 and including a titanium bead bone infiltration scaffolding defining a contiguous porous network.

FIG. 3 is a top plan view of a titanium wire infiltration scaffolding for use with the spinal fusion cage of FIG. 1.

FIG. 4 is a perspective view of the scaffolding of FIG. 3.

FIG. 5 is a partial cutaway perspective view of the scaffolding of FIG. 4 as attached to the cage of FIG. 1.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

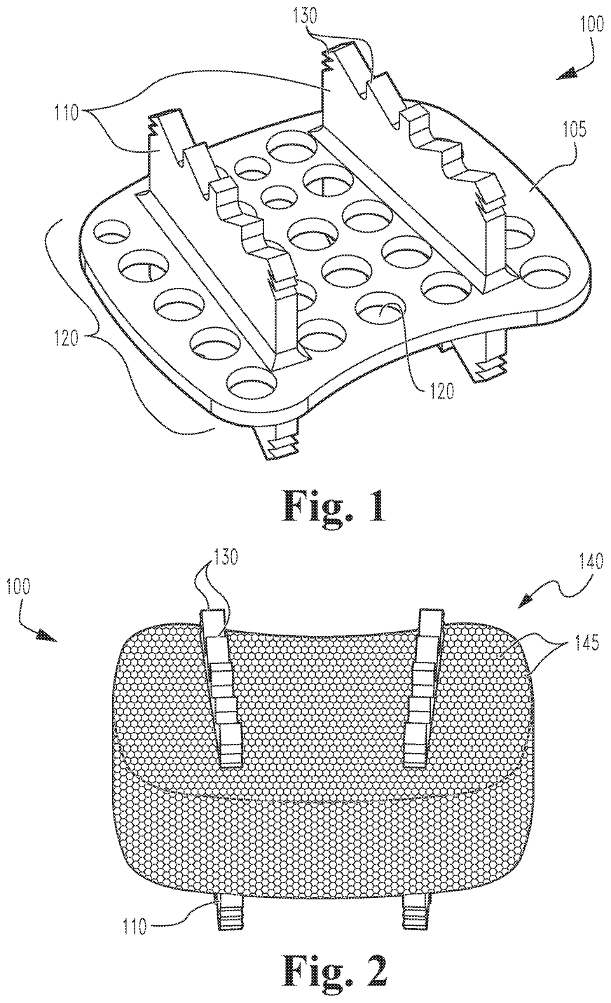

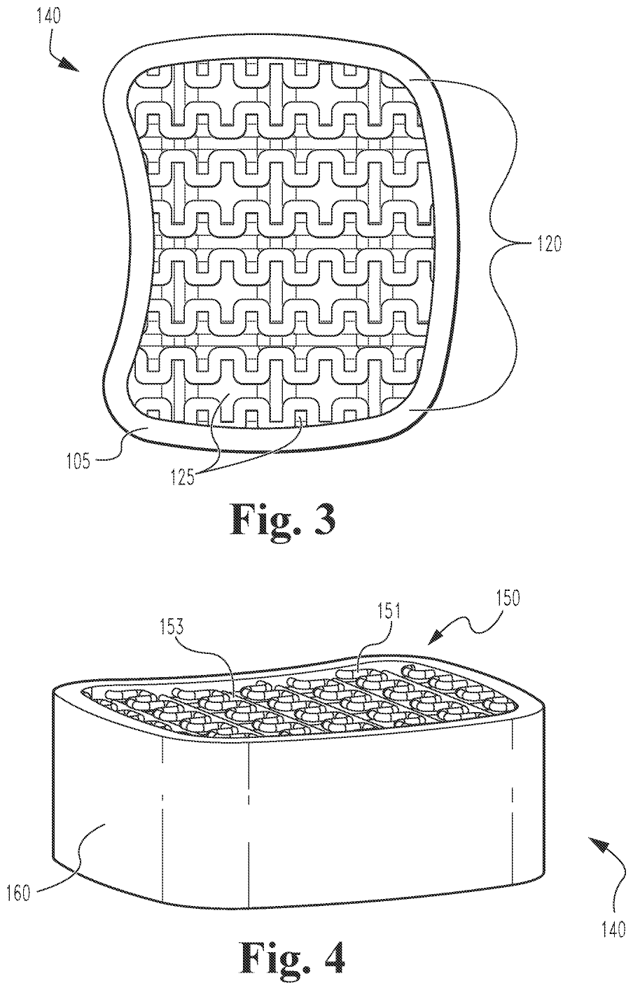

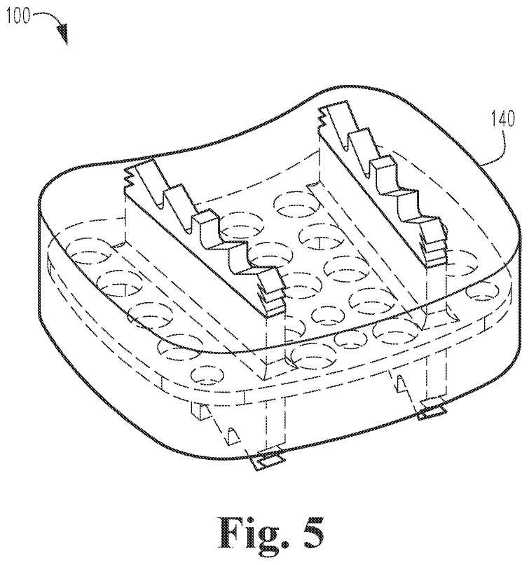

FIGS. 1-5 illustrate a first embodiment of the present novel technology, a spinal fusion cage 100 having an apertured generally flat plate member 105 with support members 110 extending therefrom. Herein, ‘generally flat’ may be taken to mean mostly flat but with contouring to match the shape of the plate 105 to the natural vertebra endplate. The generally flat plate 105 may have a central contour or dome shape to fit with the natural vertebra endplate. The fusion cage 100 may be configured for cervical, thoracic, or lumbar use. The plate member 105 is generally rectangular, and more particularly has curved edges mimicking the contours of the vertebrae between which it is to be placed. The plate member 105 includes an aperture array 120 formed therethrough, with the apertures 125 typically being of different sizes, such as including at least two different aperture areas. The pattern of the aperture array 120 may be varied, such as rows of apertures 125 oriented in a grid pattern, random placement of apertures 125, and the like.

The support members 110 extend away from the plate member for engagement with vertebrae in either direction. If the plate member 105 is assumed to be oriented horizontally, the support members 110 extend therefrom both above and below the plate member 105, typically with a vertical orientation. In other words, the support members 110 orthogonally or generally orthogonally intersect the plate member 105, with the plate member 105 bisecting the support member(s) 110.

Support members 110 include multiple teeth 130 extending therefrom for lockingly engaging bone to anchor the cage 100 in place between vertebrae. Teeth 130 are typically provided in varying sizes, with some teeth 130 angled to extend toward the plate 105 while some extend away from the plate 105 or parallel therewith. Teeth 130 are typically symmetrical along the direction of elongation, so as to minimize or avoid front/back movement. Support member 110 further includes a middle square pin 131, which is sufficiently thick so as to resist horizontal movement and/or deformation. The support members or keels 110 extend sufficiently far from the plate 105 and any adjacent filler material 140 so as to be able to penetrate the vertebral end plates of adjacent vertebrae (above and below plate 105) and remain embedded in the endplate(s) during vertical movement; the adjacent vertebral end plates are oriented parallel to plate 105. Teeth 130 are angled to intersect the vertebral end plates at an intersection angle; the intersection angle is typically between 20 and 70 degrees, more typically between 30 and 60 degrees, still more typically between 40 and 50 degrees, and yet more typically 45 degrees, so as to increase resistance to horizontal and vertical movement of the cage 100.

The plate 105 and support members 110 are typically made of a biocompatible structural material, such as titanium or natural or artificial bone, and are more typically provided as a unitary piece. 3D printed bone maybe used to make all or part of the cage 100.

A filler material 140 may be provided to extend from the plate 105 to provide support to adjacent vertebrae (when the cage 100 is emplaced therebetween) as well as to provide a porous scaffold 150 for infiltration and bone growth. In one embodiment, the filler material 140 is a volume of titanium beads 145 defining a porous scaffolding 150 disposed on both sides of the plate 105 and connected together at contact points, such as by a rapid electrical discharge welding process or the like. In this embodiment, the filler material 140 is a porous scaffolding 150 defining a solid portion (an interconnected bead array) 151 and an open cell pore network 153. The bead size and bead size distribution are variable; variations of bead size and bead size distribution allows for variation and fine-tuning of the porosity and contiguity of the open cell pore network 153. In other embodiments, the porous scaffolding 150 includes a solid portion 151 made of a twisted, interconnected wire mesh, arrays of twisted and/or curved wire, and the like.

In other embodiments, the filler 140 is made of an artificial bone material, such as hydroxyapatite, beta tricalcium phosphate, other morphologies of tricalcium phosphate, bioglass, collagen, chitosan, carbon nanotubes, combinations thereof, and the like. Artificial bone is typically provided as a solution to be 3D printed, such as by a bioprinter. Filler 140, cage 100, or both may be made of 3D printed artificial bone. In still other embodiments, the filler 140 is a combination of titanium and artificial bone. In all embodiments, the filler 140 may be infiltrated with bone growth accelerants, antibiotics, antivirals, medicines, pharmaceuticals, minerals, combinations thereof, and the like. In some embodiments, the filler 140 is surrounded by a filler wall 160. The filler wall 160 is typically made of titanium or like structural material and/or of a resorbable biocompatible material, such as bone, artificial bone, 3D printed bone/artificial bone, bioglass, printed bioglass, combinations thereof, and the like.

In operation, the device 100 is surgically placed between adjacent vertebrae once the injured or damaged disc has been removed. The teeth 130 lockingly engage the bone endplate of each respective vertebra, such that the device is essentially self-seating and does not move or shift after being emplaced. Over time, bone grows out and through the porous filler 140, permanently fusing the adjacent vertebrae. Bone grows inside the 3D printed bone 105, 110, 140 portions until the implant 100 is completely resorbed and replaced by the patient's own vertebral bone.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

What is claimed is:

1. A self-seating spinal cage, comprising:
a pair of spaced support members, each respective support member having a first end and a second end;
a plurality of teeth extending from each respective first end and each respective second end;
a plate member bisecting each respective spaced support member;
an array of apertures formed through the plate member;
a porous scaffolding operationally connected to the plate member and defining an open cell pore network, wherein the plate member bisects the porous scaffolding;
wherein the plate member and the spaced support members define a unitary structural body.

2. The self-seating spinal cage of claim 1 wherein the plate member and the support members are a 3D printed artificial bone unitary structural body.

3. The self-seating spinal cage of claim 1 wherein the porous scaffolding defines a plurality of intersecting titanium beads; and wherein each respective titanium bead is welded to an adjacent titanium bead.

4. The self-seating spinal cage of claim 1 wherein the porous scaffolding is 3D printed artificial bone.

5. The self-seating spinal cage of claim 2 wherein the artificial bone is selected from the group consisting of hydroxyapatite, beta tricalcium phosphate, other morphologies of tricalcium phosphate, bioglass, collagen, chitosan, carbon nanotubes, and combinations thereof.

6. The self-seating spinal cage of claim 1 wherein the porous scaffolding is a combination of titanium bead and artificial bone.

7. The self-seating spinal cage of claim 1 wherein the porous scaffolding is surrounded by a titanium wall.

8. The self-seating spinal cage of claim 1 wherein the porous scaffolding defines an array of titanium wires.

9. The self-seating spinal cage of claim 1 wherein the teeth are oriented to intersect a vertical end plate oriented parallel to the plate member at an angle between thirty degrees and sixty degrees.

10. The self-seating spinal cage of claim 9 wherein the teeth art oriented to intersect the vertical end plate at an angle of forty-five degrees.

11. A spinal cage assembly, comprising:
a generally flat plate member;
a first pair of spaced support members extending orthogonally from the generally flat plate member;
a second pair of oppositely disposed spaced support members extending orthogonally from the generally flat plate member;
a plurality of teeth extending from each respective support member;
an array of apertures formed through the generally flat plate member;
a porous scaffolding operationally connected to the plate member and defining an open cell pore network, wherein the plate member bisects the porous scaffolding and wherein each respective support member extends beyond the porous scaffolding;
wherein the generally flat plate member and the respective support members define a unitary structural body.

12. The spinal cage assembly of claim 11 wherein the unitary structural body is artificial bone.

13. The spinal cage assembly of claim 12 wherein the unitary body includes the porous scaffolding.

14. A method of producing a spinal cage, comprising:
a) preparing an artificial bone solution;
b) three-dimensionally printing a spinal cage device, wherein the spinal cage device further comprises:
a pair of spaced support members, each respective support member having a first end and a second end;
a plurality of teeth extending from each respective first end and each respective second end;
a plate member bisecting each respective spaced support member;
an array of apertures formed through the plate member;
a porous scaffolding operationally connected to the plate member and defining an open cell pore network, wherein the plate member bisects the porous scaffolding.
